(19) United States
(12) Patent Application Publication
(10) Pub. No.: US 2011/0218361 A1
(43) Pub. Date: Sep. 8, 2011

(54) METHOD FOR PRODUCING COMPOUND FOR PREPARATION OF ANTI-PARKINSON'S DISEASE DRUG

(75) Inventors: Chi-Hsiang Yao, Taoyuan (TW); Tsung-Ting Chen, Taoyuan (TW)

(73) Assignee: EVERLIGHT USA, INC., Pineville, NC (US)

(21) Appl. No.: 13/012,033

(22) Filed: Jan. 24, 2011

(30) Foreign Application Priority Data
Mar. 4, 2010 (TW) 099106246

(51) Int. Cl. C07C 209/22; C07C 209/68

(52) U.S. Cl. 564/428

(57) ABSTRACT

The present invention provides a method for producing a compound of formula (I), which is used for preparing anti-Parkinson's disease drugs, rasagiline and rasagiline mesylate.

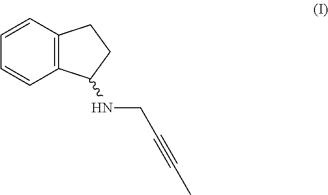

### 1. FIELD OF INVENTION

The present invention relates to a method for producing an indene derivative, and more particularly, to a method for producing a compound which is used for preparing an anti-Parkinson's disease drug, rasagiline or rasagiline mesylate.

### 2. BACKGROUND OF THE INVENTION

Rasagiline (R-(+)-N-propargyl-1-aminoindan) is a selective irreversible MAO-B inhibitor for treating Parkinson's disease. There are various disclosures of methods for preparing rasagiline. For example, Chinese Patent Application Publication No. 101062897 discloses that a reaction of 1-indanone and hydroxylamine is performed to form 2,3-dihydrogen-1H-indenes-1-ketoxime, 2,3-dihydrogen-1H-indenes-1-amine is formed, and then the reaction of the amine compound and 3-substituted propyne is performed to form rasagiline.

U.S. Pat. No. 5,994,408 discloses forming 1-chloroindan from indene, forming N-benzyl-1-aminnoidan, and performing debenzylation to form R-(−)-1-aminoindan, which is a rasagiline derivative.

Chinese Patent Application Publication No. 101260048 discloses the synthesis of rasagiline as the following scheme.

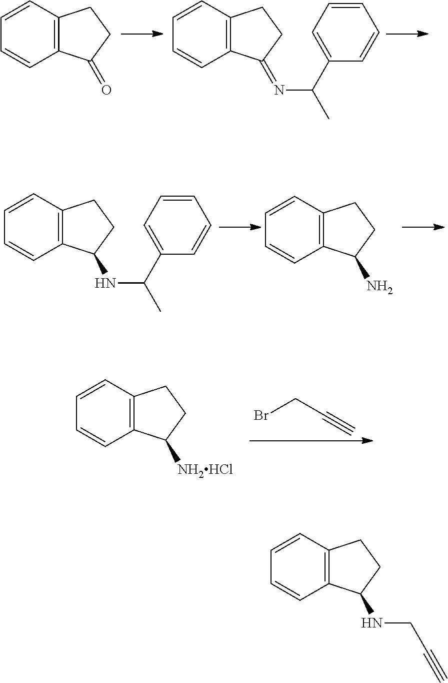

However, the above conventional methods have long synthesis pathway, results in low yield and high cost. Particularly, in Chinese Patent Application Publication No. 101260048, the selective reducing agent, (S)-(−)-α-phenylethylamino-(S)-(−)-α-methylbenzylamine is used in the reducing step at high pressure of hydrogen (3.5 bar) and 40° C. for 45 hours. The reaction time is too long, and there is a high risk due to the high pressure environment.

In addition, European Patent No. 0436492 discloses producing rasagiline from 1-amino from 1-aminoindan and 3-chloropropyne. However, the byproduct of the following formula is easily formed in the reaction.

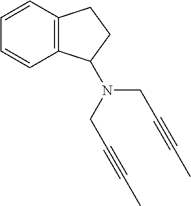

In Chinese Patent Application Publication No. 1990455, hydrogen or boron hydrides are used as a reducing agent. However, hydrogen is dangerous due to the spontaneous combustion hazard, and boron hydrides are commercially solid, which is not easy to be introduced into the reaction and even causes damages. Accordingly, there is an urgent need to provide a method for forming the therapeutic compound with simple process, good yield and low cost.

### SUMMARY OF THE INVENTION

The present invention provides a method for producing a compound of formula (I), which is used for preparing an anti-Parkinson's disease drug.

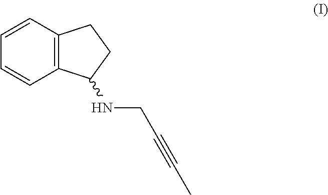

The method includes the steps of performing a reaction of 1-indanone and propargylamine to form an imine intermediate of formula (II); and

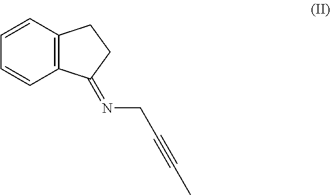

performing a reducing reaction of the imine intermediate of formula (II) and aluminum hydride in an aprotic solvent at a temperature in a range from −30 to −70° C. to obtain the compound of formula (I).

The present invention further provides a method for producing rasagiline. The method includes the steps of providing the compound of formula (I) to contact a chiral acid to form a rasagiline salt; and performing an alkalization of the rasagiline salt to form rasagiline.

In comparison with the conventional methods, the method of the present invention provides a simple and short synthesis pathway, simple operation, great yield and lower cost, and uses the safe reducing agent and materials which are easily introduced into the reaction.

### BRIEF DESCRIPTION OF THE DRAWINGS

FIG. 1 is an image showing the X-ray diffraction of the compound (III) powder according to the present invention; and

FIG. 2 is an image showing the X-ray diffraction of the rasagiline mesylate powder according to the present invention.

### DETAILED DESCRIPTION OF THE PREFERRED EMBODIMENTS

The following specific examples are used for illustrating the present invention. A person skilled in the art can easily conceive the other advantages and effects of the present invention.

The present invention provides a method for producing a compound of formula (I), which is used for preparing an anti-Parkinson's disease drug.

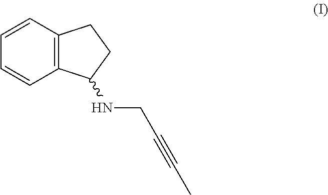

The reaction of the commercially purchased 1-indanone and propargylamine is performed in an organic solvent and catalyzed by an acid to form an imine of formula (II).

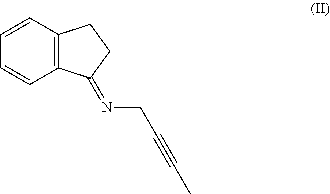

In the method of the present invention, the organic solvent may be, but not limited to, dichloromethane, methyl tert-butyl ether (MTBE), toluene, methanol or ethanol. Preferably, the organic solvent is methyl tert-butyl ether (MTBE). The acid for catalysis may be p-toluenesulfonic acid monohydrate (PTSA) or boron trifluoride etherate. Preferably, the acid is p-toluenesulfonic acid monohydrate (PTSA). The reaction may be performed at a temperature in a range from 10 to 80° C., and preferably from 20 to 30° C.

The imine of formula (II) is reduced with an aluminum hydride reducing catalyst in the aprotic solvent at low temperature, so as to form the compound of formula (I).

In the reduction reaction, the aluminum hydride reducing catalyst includes diisobutylaluminum hydride (DIBAL-H) or lithium aluminum hydride. Preferably, the aluminum hydride reducing catalyst is diisobutylaluminum hydride.

The aprotic solvent may be pentane, hexane, heptane, cyclohexane, benzene, toluene, xylene, diethyl ether, methyl tert-butyl ether or tetrahydrofuran. Preferably, the aprotic solvent is toluene or hexane.

The reduction reaction is performed at a temperature in a range from −30 to −70° C., and preferably from −40 to −60° C.

The compound of formula (I) is used for preparing anti-Parkinson's disease drugs, rasagiline and rasagiline mesylate. For example, rasagiline is obtained by the chiral separation of the compound of formula (I) with a chiral acid.

Specifically, the compound of formula (I) is mixed with and contact the chiral acid in an organic solvent, the mixture is heated till the compound of formula (I) is completely dissolved in the chiral acid, and the mixture is cool down below 20° C. The rasagiline salt of the following formula is thus obtained.

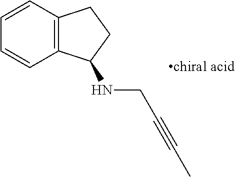

The chiral acid may be tartaric acid, malic acid or mandelic acid, and may be (+) or (−) type. Preferably, the chiral acid is mandelic acid. The organic solvent may be ethyl ether, methyl tert-butyl ether, tetrahydrofuran, or acetic ester. Preferably, the organic solvent is methyl tert-butyl ether.

The rasagiline salt is provided in a solvent such as hexane, and alkalized in a solution such as sodium hydroxide solution. After the layers are separated, the organic layer is extracted, and concentrated to obtain rasagiline.

To obtain rasagiline mesylate, rasagiline and methanesulfonic acid are provided in isopropanol, and heated to a temperature in a range from 70 to 80° C. The mixture is cool down below 20° C. to precipitate salts, which are filtered and dried, and rasagiline mesylate is thus obtained.

The synthesis of the compound of formula (I) is shown as the following scheme I, and the preparation of rasagiline and rasagiline mesylate from the compound of formula (I) is shown as the following scheme II.

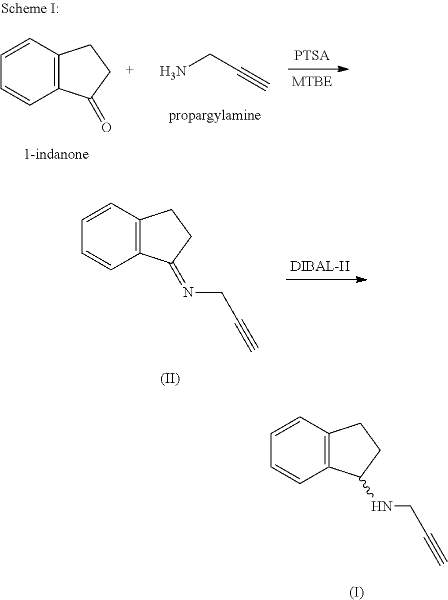

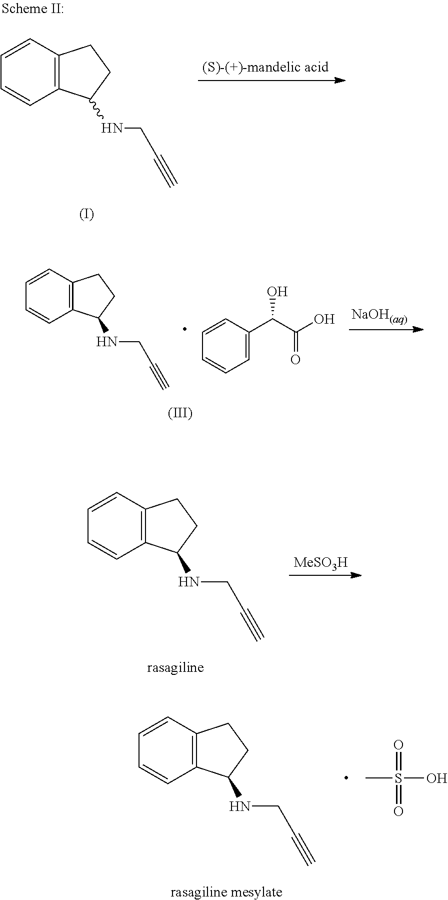

The present invention is illustrated in, but not limited to, the following embodiments. In the embodiments, the percentage is a percentage by weight, and the temperature unit is ° C. unless specified.

### Embodiment 1

### Synthesis of the Compound (I)

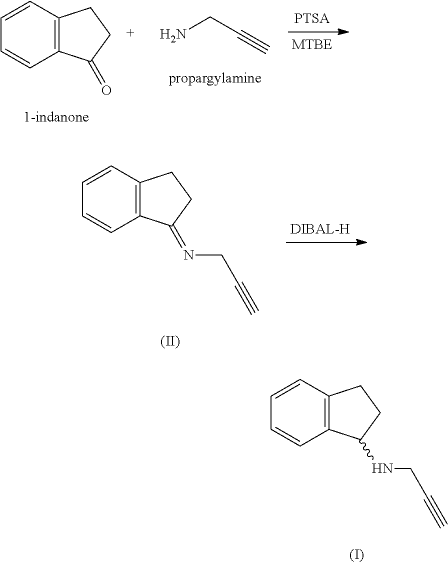

40.0 g of 1-indanone and 50.0 g of propargylamine were added in a reaction bottle, and dissolved with 40 mL of methyl tert-butyl ether (MTBE). 11.6 g of p-toluenesulfonic acid (PTSA) was added in the reaction bottle, and the mixture was stirred for 8 hours and concentrated to remove methyl tert-butyl ether. 200 mL of toluene was added in the reaction bottle and mixed with the previous concentrate. The mixture was cool down to about −70° C., and 550 mL of 20% diisobutyl aluminum hydride (DIBAL-H; dissolved in hexane) was slowly added at a reaction temperature in a range from −30 to −70° C. 1 hour later, 35 mL of acetone was added to stop the reaction.

1 L of saturated potassium sodium tartrate solution was added in the reaction bottle and the mixture was stirred for 1 hour at room temperature. The organic layer is extracted, filtered and concentrated to obtain 42.1 g of the compound (I). The total yield was 81%.

Analysis data:

Compound (II) ¹H NMR (CDCl₃):

δ: 7.87 (d, 1H), 7.27-7.44 (m, 3H), 4.26 (s, 2H), 3.10-3.14 (m, 2H), 2.76-2.81 (m, 2H), 2.27 (m, 1H)

Compound (I) ¹H NMR (CDCl₃):

δ: 7.34 (m, 1H), 7.17-7.25 (m, 3H), 4.41 (t, 1H), 3.52 (m, 2H), 2.99-3.09 (m, 1H), 2.78-2.88 (m, 1H), 2.37-2.46 (m, 1H), 2.26 (m, 1H), 1.81-1.92 (m, 1H)

### Embodiment 2

### Synthesis of the Compound (III)

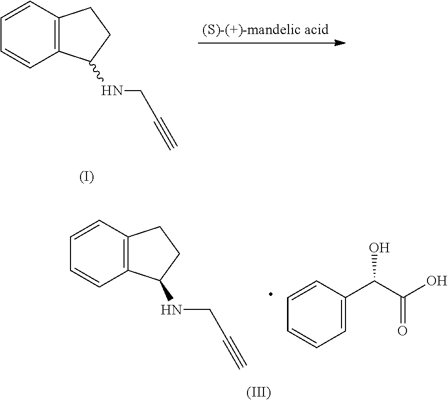

15.4 g of the compound (I) and 13.7 g of (S)-(+)-mandelic acid were added in to a reaction bottle, and mixed with 310 mL of methyl tert-butyl ether. The mixture was heated to dissolve solids. Then, the mixture was cool down to precipitate solids, and 20 hours later, the mixture was cool down below 10° C. and then maintained for 1 hour. The mixture was filtered, and the filtered solid was washed with methyl tert-butyl ether and dried, so as to obtain 13.1 g of the compound of formula (III). The yield was 45%. ¹H NMR data and X-ray diffraction data of the product were shown as follows.

¹H NMR (CDCl₃):

δ: 7.37 (m, 2H), 7.21-7.31 (m, 6H), 7.12 (m, 1H), 4.88 (s, 1H), 4.58 (dd, 1H), 3.33-3.47 (m, 2H), 2.99-3.09 (m, 1H), 2.73-2.83 (m, 1H), 2.33 (t, 1H), 2.23-2.30 (m, 1H), 1.96-2.06 (m, 1H)

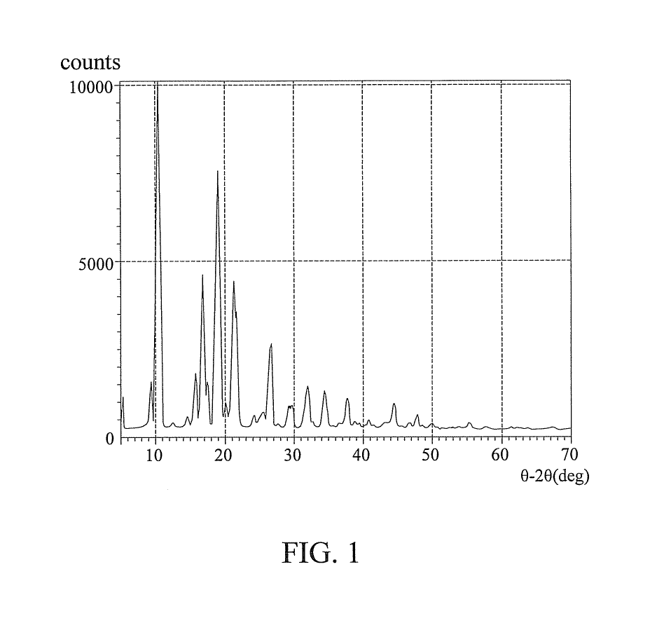

X-ray diffraction of the product:
<2Θ>=10.4±0.2, 16.8±0.2, 19.0±0.2, 21.3±0.2, 26.6±0.2
The X-ray diffraction image was shown in FIG. 1.

### Embodiment 3

### Synthesis of Rasagiline

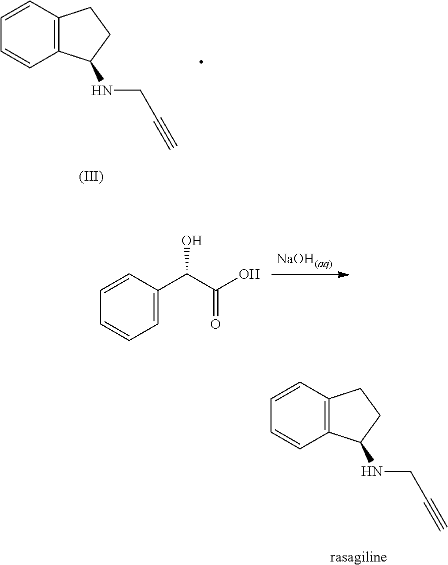

13.1 g of the compound (III) was alkalinized with 100 mL of sodium hydroxide (2%), and extracted with hexane for three times (100 mL each). The hexane layer was treated with sodium sulfate to remove water, filtered and concentrated, so as to obtain 6.2 g of rasagiline. The yield was 90%.

### Embodiment 4

### Synthesis of Rasagiline Mesylate

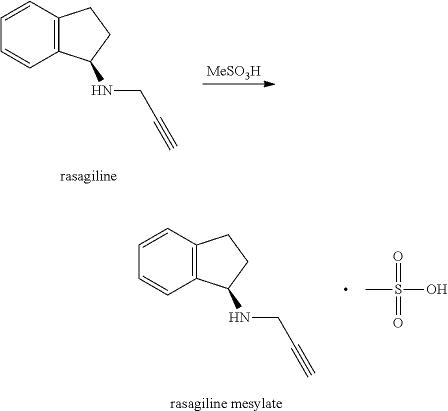

6.2 g of rasagiline was dissolved in 30 mL of isopropanol in a reaction bottle. 3.7 g of methanesulfonic acid was dropped into the reaction bottle. The mixture was heated to a temperature in a range from 70 to 75° C., to completely dissolve the solids. Then, the mixture was slowly cool down to precipitate solids. The mixture was stirred at room temperature for 16 hours, and then filtered. The filtered solid was washed with isopropanol, and dried, so as to obtain 7.7 g of rasagiline mesylate. The yield was 80%. ¹H NMR data and X-ray diffraction data of the product were shown as follows.

¹H NMR (CDCl₃):

δ: 9.35 (b, 2H), 7.65 (d, 1H), 7.23-7.37 (m, 3H), 4.95 (m, 1H), 3.86 (s, 2H), 3.22-3.30 (m, 1H), 2.85-2.95 (m, 1H), 2.54-2.57 (m, 4H), 2.42-2.50 (m, 2H)

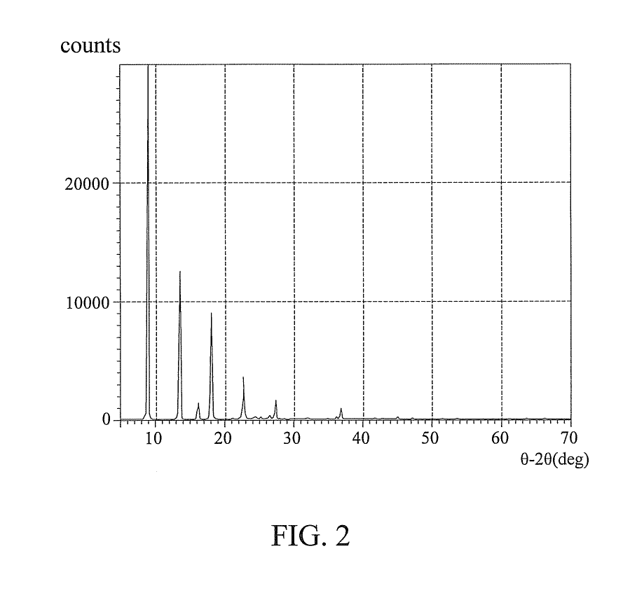

X-ray diffraction of the product:
<2Θ>=9.0±0.2, 13.6±0.2, 18.1±0.2, 22.7±0.2, 27.4±0.2
The X-ray diffraction image was shown in FIG. 2.

In the present invention, the reaction of the commercially purchased 1-indanone and propargylamine is performed in an organic solvent and catalyzed by an acid to form an imine of formula (II), and the imine of formula (II) is reduced with diisobutylaluminum hydride in the aprotic solvent at low temperature, so as to form the compound of formula (I). In the reaction, diisobutylaluminum hydride is easily controlled to be introduced into the reduction reaction. Further, in comparison with the conventional methods, the synthesis pathway in the present invention is shorter, the operation is easier and the cost is lower in the present invention.

The invention has been described using exemplary preferred embodiments. However, it is to be understood that the scope of the invention is not limited to the disclosed arrangements. The scope of the claims, therefore, should be accorded the broadest interpretation, so as to encompass all such modifications and similar arrangements.

What is claimed is:

1. A method for producing a compound of formula (I), comprising the steps of: 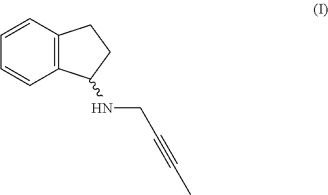
performing a reaction of 1-indanone and propargylamine to form an imine intermediate of formula (II); and 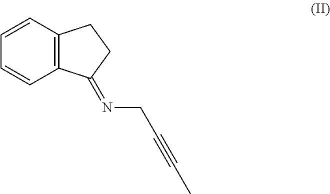
performing a reducing reaction of the imine intermediate of formula (II) and aluminum hydride in an aprotic solvent at a temperature in a range from −30 to −70° C. to obtain the compound of formula (I).

2. The method of claim 1, wherein the aluminum hydride is diisobutylaluminum hydride.

3. The method of claim 1, wherein the aprotic solvent is pentane, hexane, heptane, cyclohexane, benzene, toluene, xylene, diethyl ether, tert-butyl methyl ether or tetrahydrofuran.

4. The method of claim 1, wherein the aprotic solvent is toluene or hexane.

5. The method of claim 1, wherein the temperature is in a range from −40 to −60° C.

6. A method for producing rasagiline having a structure of 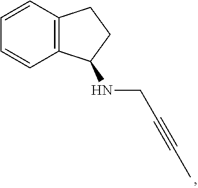
comprising the steps of:
providing the compound of formula (I) in claim 1 to contact a chiral acid to form a rasagiline salt; and
performing an alkalization of the rasagiline salt to form rasagiline.

7. The method of claim 7, wherein the chiral acid is one of tartaric acid, malic acid and mandelic acid.

8. The method of claim 6, wherein the chiral acid is mandelic acid.

9. The method of claim 6, wherein the compound of formula (I) is provided to contact the chiral acid in the presence of an organic solvent, in which the organic solvent is one of diethyl ether, tert-butyl methyl ether, tetrahydrofuran and acetic ester.

10. The method of claim 6, wherein the organic solvent is tert-butyl methyl ether.
